(19) United States
(12) Patent Application Publication
(10) Pub. No.: US 2006/0095071 A1
(43) Pub. Date: May 4, 2006

(54) Ebolic apparatus and methods for tumor vasculture system obstruction

(76) Inventor: Ping Ye Zhang, San Diego, CA (US)

Correspondence Address: Ping Ye Zhang, 11865 Aspen View Drive, San Diego, CA, 92128, US

(21) Appl. No.: 11/262,014

(22) Filed: Oct. 31, 2005

Related U.S. Application Data
(60) Provisional application No. 60/623,837, filed on Nov. 1, 2004.

(51) Int. Cl. A61M 29/00

(52) U.S. Cl. 606/200

(57) ABSTRACT

The present invention includes a method and apparatus for tumor blood vessel obstruction and tumor therapy. The embolic device is made of biocompatible materials. The embolic device may utilize thrombus-enhancing filamentary material and/or coagulate components that enhance the effectiveness to cause tumor vasculature thrombosis. The specific design of the device will facilitate tumor vasculature site space-filling. The embolic device may be implanted into the targeted tumor vasculature site by minimal invasive method.

### RELATED APPLICATION

The present application claims priority to U.S. provisional patent application, entitled EMBOLIC APPARATUS AND METHODS FOR TUMOR VASCULATURE SYSTEM OBSTRUCTION, assigned application Ser. No. 60/623,837, and filed Nov. 1, 2004.

### FIELD OF THE INVENTION

The present invention relates in general to the field of apparatus and methods for the treatment of cancer. More particularly, the present invention relates to the field of apparatus and minimum invasive methods of an implantable embolic device for tumor blood vessel obstruction and tumor therapy. Also, the embolic device may utilize thrombus-enhancing filamentary material and/or coagulate components that enhance the effectiveness to cause tumor vasculature thrombosis. The embolic device configurations are comprises of a series of overall shapes including spherical, elliptical, oval, clover or box-like. The embolic device is delivered to the targeted tumor blood vessel by a delivery system. The embolic device is then detached from the delivery system by means such as electrolysis, hydrolysis, and mechanical detachment. The implanted embolic device will obstruct vasculature system supplying tumor, therefore, to stop tumor growth and metastasis. Moreover, the application of such embolic device will lead to tumor regression and dormancy.

### DESCRIPTION OF RELATED ART

Many promising therapeutic agents have been proposed for cancer therapy for the past two decades. Their potential is proven in numerous preclinical studies. However, limited success has been achieved in solid tumor therapy. The three most common tumor treatments are surgery, radiation therapy, and chemotherapy. Surgical approach is efficient in cases of early diagnosis and smaller tumors without remote metastases. Large, advanced tumors may be removed only in rare cases, and this approach is often impossible. And tumor cell resistance to various radiation therapy and chemotherapeutic agents represents a major problem in clinical oncology for past decades. The newer approach such as photodynamic therapy (PDT), a form of energy activated therapy for destroying abnormal or diseased tissue, has received increasing interest as a mode of treatment for a wide variety of different cancers and diseased tissue (U.S. Pat. Nos. 5,445,608; 6,602,274). The first step in this therapy is carried out by administering a photosensitive compound systemically by ingestion or injection, or topically applying the compound to a specific treatment site on a patient's body, followed by illumination of the treatment site either externally or internally with light having a wavelength or waveband corresponding to a characteristic absorption waveband of the photosensitizer. The light activates the photosensitizing compound, causing singlet oxygen radicals and other reactive species to be generated, leading to a number of biological effects that destroy the abnormal or diseased tissue, which has absorbed the photosensitizing compound. The main drawbacks of external PDT methods are: (1) the risk of damage to non-target tissues, such as the more superficial cutaneous and subcutaneous tissues overlying the target tumor mass; (2) the limited volume of a tumor that can be treated; and (3) the limitation of treatment depth. Clearly, there would be significant advantage to a completely internal noninvasive form of PDT directed to subcutaneous and deep tumors, which avoids the inadvertent activation of any photosensitizer in skin and intervening tissues. However, to date, this capability has not been clinically demonstrated nor realized.

Recently, there has been much interest in the use of antiangiogenesis drugs for treating cancerous tumors by minimizing the blood supply that feeds a tumor's growth (U.S. Pat. Nos. 6,797,691; 6,632,798; 6,521,593; 6,451,312; 6,004,554). Studies have shown that the growth and metastasis of solid tumors are angiogenesis-dependent. It has been shown, for example, that tumors which enlarge to greater than about 2 mm in diameter must obtain their own blood supply and do so by inducing the growth of new capillary blood vessels. After these new blood vessels become embedded in the tumor, they provide nutrients and growth factors essential for tumor growth as well as a means for tumor cells to enter the circulation and metastasize to distant sites, such as liver, lung or bone. When used as drugs in tumor-bearing animals, natural inhibitors of angiogenesis can prevent the growth of small tumors. Indeed, in some protocols, the application of such inhibitors leads to tumor regression and dormancy even after cessation of treatment. Moreover, supplying inhibitors of angiogenesis to certain tumors can potentiate their response to other therapeutic regimens (e.g., chemotherapy). Although potentially successful in such application, this approach presents certain drawbacks. One shortcoming is that since antiangiogenesis drug is administered via a route selected from the group consisting of oral, intramuscular or intravenous, antiangiogenesis drug may harm normal tissues. And the treatment sometimes causes severe side effects that can diminish a person's quality of life.

In U.S. Pat. Nos. 6,749,853 and 5,965,132, various compositions and methods are for use in achieving specific blood coagulation of tumor vasculature, causing tumor regression, through the site-specific delivery of a coagulant. At the present time, it is generally accepted that for tumor vascular targeting to succeed, antibodies are required that recognize tumor endothelial cells but not those in normal tissues. Although several antibodies have been raised, none have shown a high degree of specificity. Also, there do not appear to be reports of any particular agents, other than the aforementioned toxins, that show promise as the second agent in a vascular targeted antibody conjugate. Thus, unfortunately, while vascular targeting presents certain theoretical advantages, effective strategies incorporating these advantages have yet to be developed.

U.S. Pat. No. 5,624,685 discloses a vascular lesion embolizing material comprising a high-polymer gel capable of absorbing water in an amount of 10 mL/g and more. When the high-polymer gel is supplied, either as such or after being bound with a binder or confined in a capsule, to the site of a blood vessel having a lesion to be repaired or its neighborhood, the gel swells upon contact with blood and spreads readily in the blood vessel to close the lumen of the blood vessels with lesion. These materials are delivered as microparticles in a carrier fluid that is injected into the vascular site, a process that has proven difficult to control. A further development in this arena has been the formulation of hydrogel materials into a preformed implant or plug that is installed in the vascular site by means such as a microcatheter (U.S. Pat. Nos. 5,258,042; 5,456,693). These types of plugs or implants are primarily designed for obstructing blood flow through a tubular vessel, and they are not easily adapted for precise implantation within other vascular structure, so as to fill substantially the entire volume of the structure.

Minimum invasive method of a purposeful delivery of a highly concentrated sugar solution to the blood supply system of a tumor directly through the patient's vascular system is described in U.S. Pat. No. 6,199,555. Because of the known sclerotic effect of concentrated sugar solutions, the blood supply system and more specifically the venous side of the tumor blood supply system collapses and ceases its function. With its blood supply suddenly and irreversibly blocked, the tumor is soon destroyed. However, the sclerotic effect of concentrated sugar solution may damage normal vascular system adjacent to the tumor. To date, this capability has not been clinically demonstrated nor realized.

It has become clear that tumors need to be vascularised to grow and metastasize. If a tumor's blood supply is curtailed, the tumor will not grow beyond 0.4 mm. Tumor cells, absent an adequate blood supply, ultimately become necrotic and/or apoptotic. Thus blood vasculature and especially new blood vessel growth or angiogenesis is an important aspect of tumor biology. In view of the drawbacks associated with previously known techniques, accordingly, what has been needed is an implantable device for obstructing tumor vasculature system, without damaging normal tissues and without severe side effects. The present invention satisfies these needs.

### BRIEF SUMMARY OF THE INVENTION

The present invention is directed to methods and apparatus for implanting embolic device into a specific area, such as tumor vasculature system. In one embodiment, the present invention system includes an embolic device, such as coil or a group of coils placed into an area of targeted tumor vasculature site. The embolic device configurations are comprises of a series of overall shapes including spherical, elliptical, oval, clover or box-like known in the art. A selection of coil shapes may be found in U.S. Pat. Nos. 4,994,069; 5,382,259; 5,304,194. The embolic device design has specific patterns to fully obstruct targeted tumor vasculature site. Materials for constructing embolic device are well known in the art and include metal materials such as stainless steel, platinum, rhodium, rhenium, palladium, tungsten, nitinol and the like, as well as alloys of these metals. Placement of the embolic device into a targeted tumor vasculature site is done via percutaneous delivery catheter. After the embolic device passes through the microcatheter and reaches the targeted tumor vasculature site, there are a number of ways to release the embolic device by means such as electrolysis (U.S. Pat. Nos. 5,122,136; 5,354,295) and mechanical detachment (U.S. Pat. Nos. 5,234,437; 5,250,071; 5,261,916; 5,304,195; 5,312,415; 5,350,397).

In an alternative embodiment, the present invention includes embolic device utilizing thrombus-enhancing filamentary material and/or coagulate components that enhance the effectiveness to cause tumor vasculature thrombosis. In this regard, it should be noted that tumor vasculature is ‘prothrombotic’ and is predisposed towards coagulation. It is thus contemplated that a targeted coagulant is likely to preferentially coagulate tumor vasculature while not coagulating normal tissue vasculature, even if other normal cells or body components, particularly, the normal endothelial cells or even stroma, express significant levels of the target molecule. This approach is therefore envisioned to be safer for use in humans, e.g., as a means of treating cancer, than that of targeting a toxin to tumor vasculature.

### BRIEF DESCRIPTION OF THE DRAWINGS

The present invention is directed to methods and apparatus for implanting embolic device into a specific area, such as tumor vasculature system, to stop tumor growth and metastasis. Moreover, the application of such embolic device will lead to tumor regression and dormancy. While the present invention is described in detail as applied to tumor vasculature system, those of ordinary skill in the art will appreciate that the present invention can be applied to other organs/sites.

FIG. 1 is a perspective view of one embodiment of the present invention in use to obstruct targeted tumor vessel upon deployment from the delivery system.

FIG. 2 illustrates perspective view of the embodiment of FIG. 1 where thrombus-enhancing filamentary material is attached to embolic device.

FIG. 3 illustrates perspective view of the embodiment of FIG. 1 coated with coagulate component.

FIG. 4 is a perspective view of the embodiment of FIG. 1 implanted into the targeted tumor vasculature site.

FIG. 5 is a perspective view of the embodiment of FIG. 1 and the embodiment of FIG. 3 implanted into targeted tumor vasculature area.

### DETAILED DESCRIPTION OF CERTAIN EMBODIMENTS OF THE INVENTION

It will be readily understood that the components of the embodiments as generally described and illustrated in the drawings herein, could be arranged and designed in a wide variety of different configurations. Thus, the following more detailed description of the embodiments of the methods and apparatus of the present invention, as represented in the drawings, is not intended to limit the scope of the invention, as claimed, but is merely representative of the embodiments of the invention.

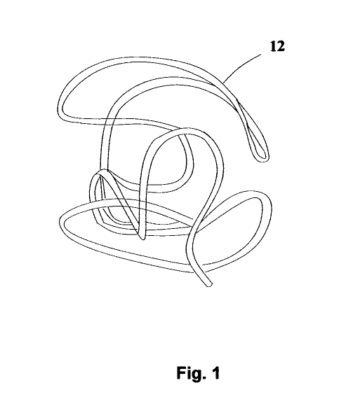

FIG. 1 illustrates a general example of embolic device to one embodiment of the present invention. The embolic device 12 forms overall shapes including spherical, elliptical, oval, clover or box-like, which fully obstructs targeted tumor vessels upon deployment from the delivery system. Preferably, the embolic device 12 may be fabricated from stainless steel, platinum, rhodium, rhenium, palladium, tungsten, nitinol and the like, as well as alloys of these metals. The embolic device 12 may be made of radiolucent fibers or polymers such as Dacron (polyester), polyglycolic acid, polylactic acid, fluoropolymers, nylon, or even silk described in U.S. Pat. No. 5,624,461. The embolic device 12 may be made of various combinations of metals and fibers to achieve desirable embolic device strength and flexibility. Those of ordinary skill in the art are knowledgeable of and will readily employ the numerous materials in the art in order to achieve the spirit of the current invention.

In a preferred embodiment of the present invention illustrated of FIG. 1, the embolic device 12 may be metal coil. The metal coils generally are constructed of a wire, usually made of a metal or metal alloy, which is wound into a helix. The metal coil has a secondary geometry or shape which dictates at least in part their space-filling occlusion mechanism. Such a secondary shape may include a secondary helical structure which involves the primary coil helix being itself wound into a second helix. In addition to the space-filling feature, another benefit to having a secondary coil shape is that it may allow the coil readily to anchor itself against the walls of a delivery site. For example, a metal coil having a secondary shape may proceed out of a sheath lumen where it was constrained in a stretched condition to have a first outer diameter equal to the sheath lumen inner diameter. When detached from a delivery system, the coil passively expands to its secondary shape, often having a larger, second outer diameter to aid in space-filling the body cavity or lumen. This may be an expansion to the coil's relaxed, unrestrained memory state—or at least until the coil encounters a vessel wall against which it exerts a force to complete the anchoring process.

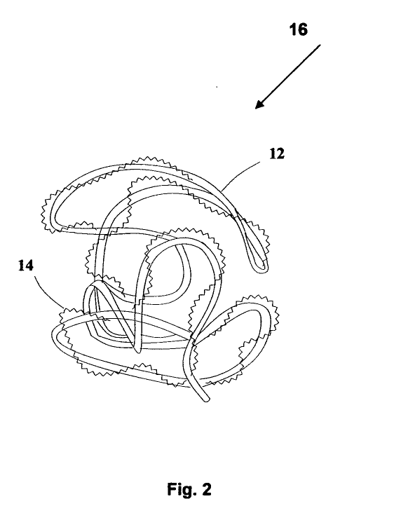

FIG. 2 illustrates partial perspective view of the embodiment of FIG. 1 where thrombus-enhancing filamentary material 14 is attached to embolic device 12, resulting filamentary material comprised embolic device 16. Use of thrombus-enhancing filamentary material is for the purpose of adding thrombogenicity to the resulting assembly. The filamentary material 14 may be attached in a variety way as described in U.S. Pat. Nos. 5,226,911; 5,382,259; 6,287,318.

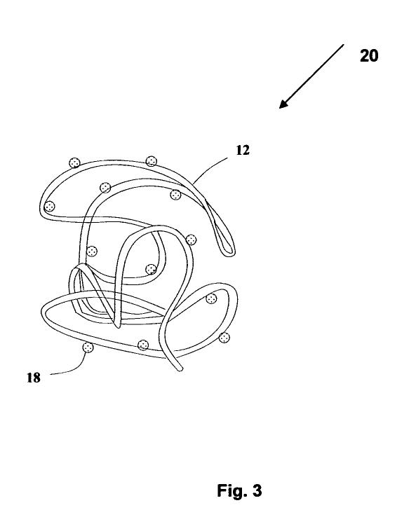

FIG. 3 illustrates perspective view of the embodiment of FIG. 1 where coagulate component 18 is coated to embolic device 12, resulting drug coated embolic device 20. Use of coagulate component 18 enhances the effectiveness to cause tumor vasculature thrombosis. The coagulate component 18 may be coated onto either exterior surface or interior surface, or both sides. Sources of coagulate component 18 on the embolic device 12 could be from drugs treating hemophilia such as ReFacto® (Wyeth), BeneFIX® (Wyeth), AlphaNine SD Coagulation Factor IX (Human) (Alpha Therapeutic Corporation). Today, hemophilia is commonly treated with products that contain concentrated amounts of the missing clotting factor. These products are called clotting factor concentrates. Two types of clotting factor products are available: plasma-derived clotting factor concentrates and recombinant clotting factor concentrates. Plasma-derived clotting factor concentrates were the first type of concentrates developed. They are produced using the plasma pooled from thousands of donors. Plasma is collected, pooled, and processed to separate the desired proteins, in this case, clotting factors. Several measures are taken to ensure the safety of all blood products, including plasma-derived clotting factor products. Blood donors are carefully screened to eliminate anyone who has been exposed to viral infections such as hepatitis or HIV; the blood used to make the clotting factor concentrates is tested for known bacterial or viral contaminants; and finally, as part of the manufacturing process, these products undergo rigorous chemical and heat treatment steps designed to inactivate viruses, rendering them harmless. These measures have improved the safety of plasma-derived clotting factor products, but have not entirely eliminated the risk of transmission of blood-borne pathogens. Recombinant clotting factor products are produced using recombinant DNA technology. The clotting factor is produced without using any human blood or cells. Recombinant clotting factors have been proven to effectively control bleeding in people with hemophilia. Clotting factor products produced using recombinant technology represent an advance in safety. Recombinant technology virtually eliminates the risk of contamination by blood-borne viruses. Recombinant products (for hemophilia as well as other therapeutic areas) have been used without transmission of blood-borne pathogens in the treatment of millions of patients throughout the world. It is desirable to incorporate coagulate component 16 into a polymer material which is then coated on the embolic device 12. The ideal coating material must be able to adhere strongly to the embolic device 12, be capable of retaining the coagulate component 18 at a sufficient load level to obtain the required dose, be able to release the drug in a controlled way over a period of time, and be as thin as possible so as to minimize the increase in profile. In addition, the coating material should not contribute to any adverse response by the body (i.e., should be non-inflammatory). The ideal coating material may be bioabsorbable materials such as poly lactic acid (PLA), polyglycolic acid (PGA), polysebacic acid (PSA), poly(lactic-co-glycolic) acid copolymer (PLGA), poly(lactic-co-sebacic) acid copolymer (PLSA), poly(glycolic-co-sebacic) acid copolymer (PGSA), polyesters, polyorthoesters, polyanhydrides, polyiminocarbonates, inorganic calcium phosphate, aliphatic polycarbonates, polyphosphazenes, collagen based adhesive, fibrin based adhesive, albumin based adhesive, polymers or copolymers of caprolactones, amides, amino acids, acetals, cyanoacrylates, degradable urethanes; or biocompatible but non-bioabsorable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina). The above disclosure is not an exhaustive list, but instead represents alternate embodiments illustrated by way of example only. Those of ordinary skill in the art are knowledgeable of and will readily employ the numerous biocompatible, biodegradable and bioerodable materials in the art in order to achieve the spirit of the current invention.

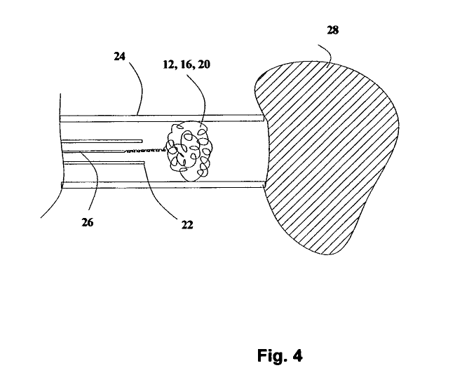

FIG. 4 depicts a common deployment method for the embolic devices described here. The first step involves the introduction of the microcatheter 22 to the targeted tumor vasculature site 24 that provides blood supply to the tumor 28. The microcatheter 22 commonly tracks a guide wire to a point just proximal of or within the desired site for occlusion. The second step involves delivering the embolic device through the microcatheter 22. As embolic device continues to extend from the catheter, it will become more convoluted and will form an occlusive site within vessel 24. Embolic device is thereafter detached from the delivery system 26 by means well known in the art including electrolytic detachment (U.S. Pat. Nos. 5,122,136; 5,354,295), mechanical detachment (U.S. Pat. Nos. 5,234,437; 5,250,071; 5,261,916; 5,304,195; 5,312,415; 5,350,397). Once an embolic device is implanted at a desired site, occlusion results either from the space-filling mechanism inherent in the device itself, or from a cellular response to the device such as a thrombus formation, or both.

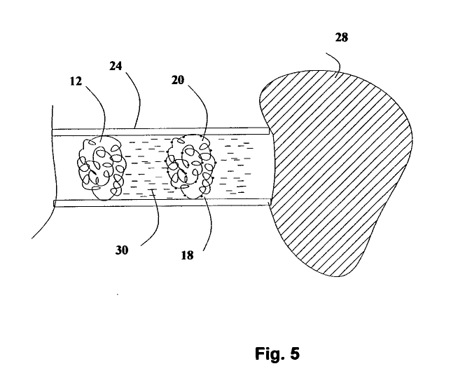

FIG. 5 is a perspective view of embolic device 12 and drug coated embolic device 20 placed into targeted tumor vasculature area 24. Preferably, one or more devices 20 are placed into the targeted tumor blood vessel area 24 followed by subsequent one or more devices 12 placement in adjacent. Tumor vasculature thrombosis 30 caused by coagulate component 18 coated on device 20 is not able to migrate through the obstruction created by embolic device 12 placement, thus, the normal blood vessels adjacent are not affected by such thrombosis.

What is claimed is:

1. An implantable embolic device comprising a structure for tumor vasculature system obstruction.

2. The device of claim 1 comprising an implantable member having two opposite ends, and wherein said member forms
a. first shape having a first outer diameter when readily constrained for minimal invasive delivery into a targeted tumor vasculature site and
b. second shape having second outer diameter larger than said first outer diameter and sufficient to space-fill the targeted tumor vasculature site.

3. The device of claim 1 comprising filamentary material to add thrombogenicity to the resulting assembly.

4. The device of claim 1 coated with coagulate component to enhance the effectiveness to cause tumor vasculature thrombosis.

5. The device of claim 1, wherein said device further comprises a releasable attachment mechanism.

6. The device of claim 1, wherein the structure is compatible with the target location of a mammalian body.

7. The device of claim 3, wherein the structure and filamentary material are compatible with the target location of a mammalian body.

8. The device of claim 1, wherein the materials are selected from the group consisting of biologically inert materials such as stainless steel, platinum, rhodium, rhenium, palladium, tungsten, nitinol and the like, as well as alloys of these metals.

9. The device of claim 3, wherein the filamentary materials are selected from the group consisting of biocompatible materials such as Dacron (polyethyleneterephthalate), polyglycolic acid, polyactic acid, fluoropolymer (polytetrafluoroethylene), nylon (polyamide), or silk.

10. The device of claim 4, wherein the coagulate components are selected from the group consisting of blood clotting products such as ReFacto® (Wyeth), BeneFIX® (Wyeth), AlphaNine SD Coagulation Factor IX (Human) (Alpha Therapeutic Corporation).

11. The device of claim 4, wherein the coating materials are selected from the group consisting biocompatible and/or biodegradable materials such as poly lactic acid (PLA), polyglycolic acid (PGA), polysebacic acid (PSA), poly(lactic-co-glycolic) acid copolymer (PLGA), poly(lactic-co-sebacic) acid copolymer (PLSA), poly(glycolic-co-sebacic) acid copolymer (PGSA), polyesters.

12. The method for delivering the device of claim 1 to a target location within a mammalian body comprising:
introducing said device via a catheter based system;
delivering said device to targeted area;
releasing said device to targeted area;
allowing said device to obstruct said targeted area.

13. The method of claim 12, wherein the step for releasing the said device is selected from the group such as mechanical detachment, electrolytic detachment and vacuum pressure separation.

14. The method for delivering the device of claim 3 to a target location within a mammalian body comprising:
attaching filamentary materials to said device;
introducing said device via a catheter based system;
delivering said device to targeted area;
releasing said device to targeted area;
allowing said device to obstruct said targeted area.

15. The method of claim 14, wherein the step for releasing the said device is selected from the group such as mechanical detachment, electrolytic detachment and vacuum pressure separation.

16. The method for delivering the device of claim 4 to a target location within a mammalian body comprising:
adhering coagulate component formula to said device;
introducing said device via a catheter based system;
delivering said device to targeted area;
releasing said device to targeted area;
allowing said device to obstruct said targeted area.

17. The method of claim 16, wherein the step for releasing the said device is selected from the group such as mechanical detachment, electrolytic detachment and vacuum pressure separation.

18. A method of treating tumor comprising:
introducing one or more said device of claim 1 via a catheter based system;
delivering said device(s) to targeted area;
releasing and placing said device(s) at targeted area;
allowing said device(s) to optimally obstruct said vasculature system supplying blood to tumor, therefore, to stop tumor growth and metastasis. Moreover, the application of such said device(s) will lead to tumor regression and dormancy.

19. A method of treating tumor comprising:
introducing one or more said device of claim 3 via a catheter based system;
delivering said device(s) to targeted area;
releasing and placing said device(s) at targeted area;
allowing said device(s) to optimally obstruct said vasculature system supplying blood to tumor; allowing said device(s) to add thrombogenicity into the said targeted area, therefore, to stop tumor growth and metastasis. Moreover, the application of such said device will lead to tumor regression and dormancy.

20. A method of treating tumor comprising:
introducing one or more said device of claim 4 via a catheter based system;
delivering said device(s) to targeted area;
releasing and placing said device(s) at targeted area;
allowing said device(s) to optimally obstruct said vasculature system supplying blood to tumor; allowing said device(s) to cause thrombosis into the said targeted area, therefore, to stop tumor growth and metastasis. Moreover, the application of such said device(s) will lead to tumor regression and dormancy.

21. A method of treating tumor comprising:
introducing one or more embolic device via a catheter based system;
delivering said device(s) to targeted area;
releasing and placing said device(s) at targeted area;
allowing said device(s) to optimally obstruct said vasculature system supplying tumor; allowing said device(s) to add thrombogenicity into the said targeted area, therefore, to stop tumor growth and metastasis. Moreover, the application of such said device(s) will lead to tumor regression and dormancy;
Subsequently,
introducing one or more embolic device via a catheter based system;
delivering said device(s) to target area;
releasing and placing said device(s) adjacent to previously implanted embolic device; allowing said device(s) to optimally obstruct said vasculature system supplying tumor, therefore, to stop tumor growth and metastasis. Moreover, the application of such said device(s) will lead to tumor regression and dormancy; Furthermore, the application of such said device(s) will prevent thrombosis from migrating to the normal blood vessel adjacent to the targeted tumor vasculature.

22. A method of treating tumor comprising:
introducing one or more embolic device via a catheter based system;
delivering said device(s) to targeted area;
releasing and placing said device(s) at targeted area;
allowing said device(s) to optimally obstruct said vasculature system supplying tumor; allowing said device(s) to cause thrombosis into the said targeted area, therefore, to stop tumor growth and metastasis. Moreover, the application of such said device(s) will lead to tumor regression and dormancy;
Subsequently,
introducing one or more embolic device via a catheter based system; delivering said device(s) to targeted area;
releasing and placing said device(s) adjacent to previously implanted embolic device;
allowing said device(s) to optimally obstruct said vasculature system supplying tumor, therefore, to stop tumor growth and metastasis. Moreover, the application of such said device(s) will lead to tumor regression and dormancy; Furthermore, the application of such said device(s) will prevent thrombosis from migrating to the normal blood vessel adjacent to the targeted tumor vasculature.
